(19) United States
(12) Patent Application Publication
(10) Pub. No.: US 2016/0184188 A1
(43) Pub. Date: Jun. 30, 2016

(54) Dental Restoration Putty and Method of Formulating and Applying

(71) Applicant: Robert B. Hankins, Del Valle, TX (US)

(72) Inventor: Robert B. Hankins, Del Valle, TX (US)

(21) Appl. No.: 14/584,637

(22) Filed: Dec. 29, 2014

(51) Int. Cl. A61K 6/02; A61K 6/027; A61K 6/097

(52) U.S. Cl. CPC A61K 6/02; A61K 6/097; A61K 6/0205; A61K 6/027

(57) ABSTRACT

A dental restoration putty and method of fabricating and allying the dental restoration is used to repair/replace a damaged or missing section of an individual's tooth or other area of dentition. The putty that is produced and utilized is an admixture of a colorant with a base material. The colorant of the putty functions to alter the color/appearance of a dental restoration to accurately match the color of an individual's existing tooth or teeth.

This application is a continuation-in-part (C.I.P.) of application Ser. No. 13/543,853 with a filing date of Jul. 8, 2012 now abandoned.

### TECHNICAL FIELD

The invention generally pertains to dental restoration products, and more specifically to a putty utilized to fabricate a dental restoration and a method of formulating and applying of the putty material.

### BACKGROUND ART

Putty materials, often referred to as peg putties, have long been used in a dental laboratory and increasingly in a dental operatory during the manufacture of dental restorations. White peg putty is placed, for example, in bridges and on veneers and the like with a peg or pin inserted into the putty prior to firing to harden the putty. The restoration, which is typically a bridge or veneer, can then be easily handled while the restoration is stained, glazed or subjected to any other appropriate operation.

Die putty which is made of a composite material has been in industry-wide use for a number of years. Typically, the putty is colored to match the color of a person's dentition. The prior art has also contemplated various procedures for determining color and shade for tooth and dental restorations. One example discloses a shade determination apparatus and methodology useful in the practice of clinical dentistry and in a dental laboratory to permit a tooth or a dental restoration to be colored. The prior art illustrates the attention previously devoted in the industry to matching the coloration and shading of prosthetic teeth and dental restorations to existing teeth with which such restorations function.

It is important to note the differences between various types of putty. There is no comparison between white putty and colored putty. For the instant invention the comparison is between a colored putty that can be fired (heated) at up to three thousand degrees, and a colored composite material that cannot be fired.

Additionally, a color/shaded putty that is placed inside a dental restoration does not have to be removed before completing a fired, colored/stained restoration. A colored or non-colored composite material must be removed before firing a restoration or the composite material will burn within a furnace.

A search of the prior art did not disclose any literature or patents that read directly on the claims of the instant invention. However, the following U.S. patents are considered related:

| PAT. NO. | INVENTOR | ISSUED |
|---|---|---|
| 6,743,014 | Kerschbaumer, et al | 1 Jun. 2004 |
| 6,623,271 | Pruden | 23 Sep. 2003 |
| 6,499,998 | Kerschbaumer, et al | 31 Dec. 2002 |

The U.S. Pat. No. 6,743,014 patent discloses a shade determination apparatus for teeth and dental restorations. A set of reference templates for comparing to a patient's tooth is provided. Based on the coloration of the reference template, the shade of a tooth or of a restoration to be employed can be determined. A storage apparatus stores the coloration of the reference templates, and the layering of the tooth or of the dental restoration can be matched and determined based on partial images form an image of the patient's tooth.

The U.S. Pat. No. 6,623,271 patent discloses a mouth simulator apparatus that simulates a patient's atmosphere of the oral environment so that a dental restorative material can be viewed after manufacture and prior to insertion in the patient's mouth. One is able to view the dental restorative material in comparison to shade guide components under the same lighting conditions as present in the oral environment.

The U.S. Pat. No. 6,499,998 patent discloses a shade determination apparatus for teeth and dental restorations. A set of reference templates for comparing to a patient's tooth is provided, whereby based on the coloration of the reference template the shade of a tooth or of a restoration to be employed can be determined. The reference templates are produced in a layer arrangement, taking into consideration layer thickness and/or material selection, that corresponds to the tooth or the dental restoration to be employed.

For background purposes and indicative of the art to which the invention relates, reference may be made to the following remaining patents found in the patent search.

| PAT. NO. | INVENTOR | ISSUED |
|---|---|---|
| 6,575,751 | Lehmann, et al | 10 Jun. 2003 |
| 6,031,209 | Panzera, et al | 29 Jan. 2000 |
| 5,800,164 | Pfau | 1 Sep. 1998 |
| 5,795,372 | Leon | 10 Mar. 1998 |
| 5,690,486 | Zigelbaum | 25 Nov. 1997 |
| 5,240,414 | Thompson | 31 Aug. 1993 |
| 4,836,674 | Lequime, et al | 6 Jun. 1989 |
| 4,654,794 | O'Brien | 31 Mar. 1987 |

### DISCLOSURE OF THE INVENTION

The invention discloses a dental restorative putty material and a method of use of the putty. During a dental restoration, the putty, which is sometimes referred to as a colored peg putty is produced in a variety of colors or shades that relate to shade guides used in the dental industry. One or more of the colored putty materials is employed in the same manner as prior uncolored peg putties during the manufacture of a dental restoration. The instant putty and method allows a practitioner to more effectively color, stain or otherwise shade a restoration to duplicate the natural tooth color of an individual who requires a dental restoration. Use of the colored putty material typically involves selection of one colored material from a set of differently colored materials to color match an individual's tooth stump having a color matching the individual's other tooth or teeth. Use of the instant colored putty facilities coloring a restoration with minimal time required to accurately color the restoration. The instant colored putty can be used with all prior materials commonly employed for manufacture of restorations including porcelains, zirconia and ceramic material as well as lithium silicates and disilicates including monolithic species thereof and with processes employed not only in a dental laboratory but also in the dental operatory whether conventional or with computer-aided techniques.

The invention provides an all-in-one putty material and a stump guide for facilitating the staining of all restorations, and especially highly translucent restorations.

The invention also provides a colored putty material useful for cracked or broken pressables, jacket crowns, veneers and the like, as well as for procedures including soldering, or mounting of copings on pegs, thereby allowing for little or no lingual band and for staining and re-glazing of veneers and the like to maintain marginal and internal integrity.

In view of the above disclosure the primary object of the invention is to provide an improved colored putty material and method of use that is utilized for all types of dental restoration that require putty.

In addition to the primary object it is also an object of the invention to provide a colored putty material and method of use that:
can be accurately color-matched to a person's teeth,
can be used for adults and children,
is safe,
is long-lasting and durable,
significantly improves on prior art dental restoration materials(s),
does not have any taste, and
can be used for a variety of restoration procedures.

These and other objects and advantages of the present invention will become apparent from the sub sequent detailed description of the preferred embodiment and the appended claims taken in conjunction with the accompanying drawings.

### BRIEF DESCRIPTION OF THE DRAWINGS

FIG. 1 is a side elevational view of a damaged tooth in need of a dental restoration.

FIG. 2 is a side elevational view of an individual's tooth having a non-colored dental restoration that does not match the color of the individual's existing tooth.

FIG. 3 is a side elevational view of an individual's tooth having a colored dental restoration that matches the color of the individual's existing tooth.

### BEST MODE FOR CARRYING OUT THE INVENTION

The best mode for carrying out the invention is presented in terms that disclose a preferred embodiment, with multiple color/shade configurations, of a dental restoration putty and method of formulating and applying 10 thereof.

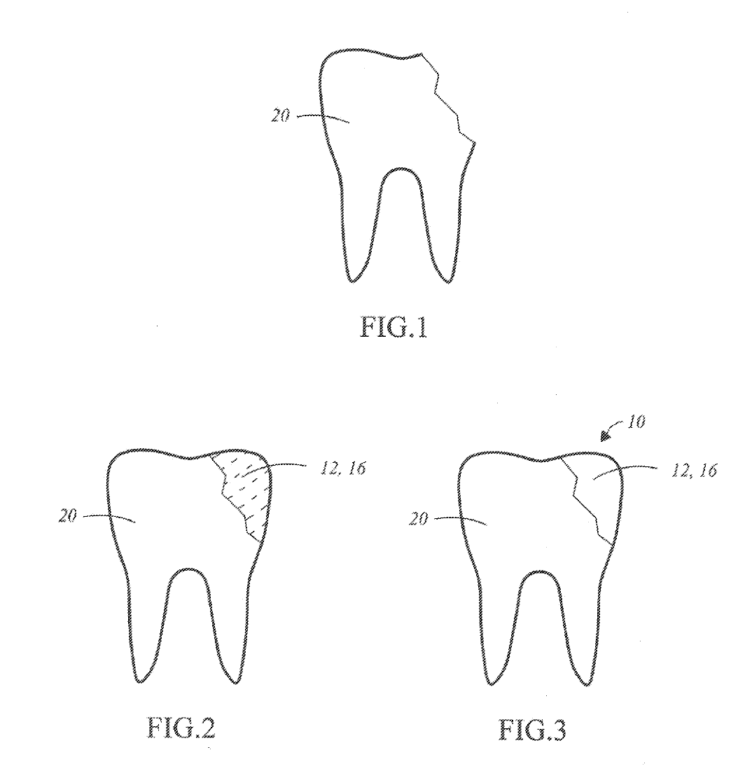

When an individual requires a dental restoration procedure, which includes for example tooth or teeth replacement, a crown or an inlay, a putty material also known as peg putty is typically utilized. The putty is also known as a peg putty because it is often used along with a support peg or pin that maintains a dental restoration correctly oriented and in place. When a dental restoration is applied a common problem that occurs is any existing/actual part of an individual's damaged tooth 20 will be discolored. When conventional, non-colored putty 14 (in the form of a dental restoration) is applied, the color of the existing/actual tooth 20 will not match the color of the putty 14, thereby allowing the restoration to be clearly visible, as shown in FIG. 2. In order to insure that the dental restoration appears as natural as possible, a dental restoration is typically color-matched to the individual's actual/other teeth, as shown in FIG. 3. The color material is accomplished by use of a colorant in the form of a stain and/or a glaze that is applied onto the restoration during the manufacture/creation of the restoration. The instant invention, as shown in FIGS. 1-3, discloses an improved putty material and method of formulating and applying the putty for dental restorations.

The putty material (putty) 14 that is produced is an admixture of colorant comprising a base material and a colorant component. The dental restoration 12 is also known as a translucent ceramic restoration and is made of a material selected from the group consisting of lithium disilicates, lithium silicates, Felspathic, Zirconia and Lucite.

The base material is comprised of a refractory ceramic fibrous material that itself is comprised of water (H₂O) in a range of 50 to 70 percent by weight refractories, aluminum oxide, and quartz hydroxyethylcelluose in a range of one to three percent by weight.

The colorant that is used to produce a specific color/shade of an individual's tooth color, as shown in FIG. 3, is comprised of an admixture of organic colors including food colors, or an admixture of inorganic colors including food grade metallic oxides.

As previously disclosed, the putty 14 is utilized to fabricate a dental restorative element. Typically, the element is comprised of a dental restoration 12 in the form of a translucent ceramic restoration that is color-matched to an individual's existing dentition, such as a damaged tooth 20, as shown in FIG. 1.

The method for allying the putty to an individual's dental restoration comprises the following steps:
a) select an individual who has a damaged tooth (teeth) 20 that requires a dental restoration procedure,
b) produce a colored putty having a color/shade that corresponds to the color/shade of the individual's existing/actual tooth or teeth,
c) press a portion of the colored putty into the individual's dental crown, an internal area of an inlay, an onlay, a rear surface, a front surface or a side surface of the tooth, or other selected area of the individual's restoration,
d) insert at least one firing peg into any exposed colored putty, insure that the firing peg is dimensioned to allow a dental practitioner to hold the peg without his/her fingers touching the colored putty of the dental restoration, add ceramic stains where necessary to make the restoration's appearance match the patient's actual tooth, and a glaze may also be applied at this time or later,
e) place the dental restoration, while still attached to the firing peg, onto a firing to tray and into a furnace and fire to the furnace's manufacturer's suggested temperature to produce a color that is a permanent component of the restoration,
f) remove the restoration and the firing tray from the furnace and allow them to cool until they are safe to handle,
g) remove any remaining colored putty from the restoration, and
h) attach restoration to the individual's dentition.

Additionally, there can be a step of adding a ceramic stain to a colored restoration to insure that the restoration accurately resembles the individual's existing tooth or teeth. Further, to insure that no superfluous colored putty remains on the restoration, sandblasting the restoration can be performed in addition to the typical cleaning/wiping the restoration prior to use.

Putty placed on a restoration that uses a peg or pin permits handling of the restoration through the agency of the peg. The peg is positioned in the putty to avoid contact with any portion of a restoration such as a veneer to prevent the color of the peg from being visible through the restoration. The restoration can then be stained, or glazed to attain the correct shade. After staining or glazing, the restoration is conventionally glazed and heat treated followed by removal of the hardened putty which has hardened after any firing step.

It should be noted that while the use of at least one firing peg is preferred, a firing peg is not required. For certain applications the putty 14 can be applied without a firing peg and will function with complete efficacy.

Also the colored putty can be packaged as a kit with colors typically useful to a dental practitioner, and/or a kit optionally including other materials or components that are useful to a dental practitioner.

An example of a formulation of the colored putty 14 is ivory and is formulated by the addition of 4.25 drams of an ivory colorant which is a combination of primary and secondary colors with one gallon of base material such as the refractory ceramic fibrous material disclosed herein. The ivory colorant is a type of food coloring produced by Jack Guttman, Inc. of West Chester, Ohio under the product designation Ivory 136BOB, IP-5228. Other food colorings available from alternate sources can also be utilized as can colorants of differing type. The colorants include low or no toxicity, the ability to mix with a chosen base material and the ability to burn away along with the base material when necessary. Other colors or combinations of colors can be produced by adding various amounts of different food colors to the base material.

While the invention has been described in detail and pictorially shown in the accompanying drawings it is not to be limited to such details, since many changes and modification may be made to the invention without departing from the spirit and the scope thereof. Hence, it is described to cover any and all modifications and forms which may come within the language and scope of the claims.

1. A dental restoration putty and method of formulating and applying, wherein said putty is utilized to produce a dental restoration that is comprised of a base material that is admixed with a colorant to match the color of said dental restoration to a shade of dentition within a person's mouth.

2. The dental restoration putty as specified in claim 1 wherein said dental restoration is comprised of refractory ceramic fibers.

3. The dental restoration putty as specified in claim 1 wherein said base material is comprised of a refractory ceramic fibrous material.

4. The dental restoration putty as specified in claim 3 wherein the refractory ceramic fibrous material is comprised of alumino-silicate wools.

5. The dental restoration putty as specified in claim 3 wherein said base material is comprised of H₂O, refractories, aluminum oxide, and quartz hydroxyethylcellulose.

6. The dental restoration putty as specified in claim 5 wherein the quantity of H₂O is 50 to 70 percent by weight.

7. The dental restoration putty as specified in claim 5 wherein the quantity of quartz hydroxyethylcelluose is in a range of one to three percent by weight.

8. The dental restoration putty as specified in claim 1 wherein said colorant is comprised of an admixture of organic colors.

9. The dental restoration putty as specified in claim 8 wherein the organic colors are comprised of food colors.

10. The dental restoration putty as specified in claim 1 wherein said colorant is comprised of an admixture of inorganic colors.

11. The dental restoration putty as specified in claim 10 wherein the inorganic colors are comprised of metallic oxides.

12. The dental restoration putty as specified in claim 1 wherein said putty material is placed inside or behind a translucent ceramic restoration to represent the actual color of an individual's tooth as the restoration is stained and glazed.

13. A dental restoration putty and method of formulating and applying that is produced and utilized as an admixture of colorant in the fabrication of a dental restorative element, which is also known as a dental restoration, wherein said colored putty material matches a shade of an individual's tooth or other dentition, wherein said putty is comprised of a base material and a colorant, wherein the dental restoration is made of materials selected from the group consisting of lithium disilicates, lithium silicates, Felspathic, Zirconia and Lucite.

14. The dental restoration putty as specified in claim 13 wherein said dental restorative element is comprised of a translucent ceramic restoration.

15. The dental restoration putty as specified in claim 13 wherein said base material is comprised of a refractory ceramic fibrous material.

16. The dental restoration putty as specified in claim 13 wherein said base material is comprised of H₂O in a range of 50 to 70 percent by weight, refractories, aluminum oxide, and quartz hydroxyethylcelluose in a range of one to three percent by weight.

17. The dental restoration putty as specified in claim 13 wherein the colorant is comprised of an admixture of organic colors, including food colors, to produce a specified shade of an individual's tooth color.

18. The putty material as specified in claim 13 wherein the colorant is comprised of an admixture of inorganic colors, including metallic oxides, to produce a specific shade of an individual's tooth color.

19. The putty material as specified in claim 14 wherein said putty material is placed inside or behind the translucent ceramic restoration to produce an actual color of an individual's tooth as the restoration is stained and glazed.

20. A method for applying a colored putty to an individual's dentition, wherein said method comprises the following steps:
a) select an individual who has a damaged tooth (teeth) that requires a dental restoration procedure,
b) produce a colored putty having a color/shade that corresponds to the color/shade of the individual's existing/actual tooth or teeth,
c) press a portion of the colored putty into the individual's dental crown, an internal area of an inlay, an onlay, a rear surface, a front surface or a side surface of the tooth, of the individual's dental restoration, or other selected area of the individual's restoration,
d) insert at least one firing peg into any exposed colored putty, insure that the firing peg is dimensioned to allow a dental practitioner to hold the peg without their fingers touching the colored putty of the dental restoration, add ceramic stains where necessary to make the restoration's appearance match the patient's actual tooth, and a glaze may also be applied at this time or later,
e) place the dental restoration, while still attached to the firing peg, onto a firing tray and into a furnace and fire to the furnace's manufacturer's suggested temperature to produce a color that is a permanent component of the restoration,
f) remove the restoration and the firing tray from the furnace and allow them to cool until they are safe to handle,
g) remove any remaining colored putty from the restoration, and
h) attach restoration to the individual's dentition.

21. The method as specified in claim 20 further comprising a step of adding additional ceramic stain to insure that the restoration accurately resembles the individual's existing tooth or teeth.

22. The method as specified in claim 21 further comprising a step of sandblasting the restoration to insure no superfluous colored putty remains after step h).
